(19) United States
(12) Patent Application Publication
(10) Pub. No.: US 2009/0326438 A1
(43) Pub. Date: Dec. 31, 2009

(54) Method for sterilizing and disinfecting body tissues by ising iontophoresis and the medical device

(76) Inventor: Nobuyoshi Karashima, Oita-City (JP)

Correspondence Address: KUBOVCIK & KUBOVCIK, SUITE 1105, 1215 SOUTH CLARK STREET, ARLINGTON, VA, 22202, US

(21) Appl. No.: 12/585,107

(22) Filed: Sep. 3, 2009

Related U.S. Application Data
(62) Division of application No. 10/800,914, filed on Mar. 16, 2004.

(30) Foreign Application Priority Data
Mar. 20, 2003 (JP) 2003-078236

(51) Int. Cl. A61N 1/30

(52) U.S. Cl. 604/20; 604/501

(57) ABSTRACT

An iontophoresis-based medical device comprising a positive electrode section, a negative electrode section, a power source supplying an electric current to the positive electrode section and negative electrode section and a controller controlling the current value and conduction time of the electric current, the iontrophoresis-based medical device for allowing the drug solution to permeate into a lesion based on ionphoresis obtained by conducting current between the positive electrode section and negative electrode section, wherein the positive and negative electrode section are respectively given a handleable stick shape, the positive electrode section is provided with a drug solution retainer which retains the drug solution and contacts with a lesion, and the negative electrode section is provided with a solution retainer which retains the solution and contacts with a part of the body in the vicinity of the lesion.

This application is a division of application Ser. No. 10/800,914, filed Mar. 16, 2004, which claims priority based on Japanese Patent Application No. 2003-078236, filed Mar. 20, 2003, and which are incorporated herein by reference.

### BACKGROUND OF THE INVENTION

1. Field of the Invention

This invention relates to a method for sterilizing and disinfecting body tissues by using iontophoresis and the medical device thereof, more particularly, a method for sterilizing and disinfecting body tissues to treat a part affected by a pathogen, for example, by using a drug solution permeated into the lesion by means of iontophoresis and the medical device thereof.

2. Description of the Prior Art

A conventional iontophoresis-based medical device for sterilizing and treating a tooth infected with a pathogenic organism by iontophoresis is known, for example, as the device claimed in the Patent literature 1.

The conventional iontophoresis-based medical device was provided with an electric circuit having a voltage generator and a current-supplied application apparatus, a positive electrode section and a negative electrode section, wherein the positive electrode section was provided needle-shaped and deeply inserted into a tooth duct and the negative electrode section was directly attached to a part of a patient body, and the voltage generator fed a direct current and the electric circuit was provided with an apparatus to maintain a constant direct current while an impedance of the electric circuit varied, and the impedance was determined depending on a body part of a patient through which a current passed.

In sterilizing and treating a tooth, a drug solution applied to the needle-shaped positive electrode section was inserted into a tooth duct, and the negative electrode section was attached to a part of a patient (for example, the wrist) to effect electric conduction. This process made it possible to provide a closed electric circuit among the power source, the electric circuit, the positive and negative electrode sections, the tooth and the part of the patient, thereby allowing permeation of the drug solution deeply into the tooth duct by iontophoresis to sterilize and treat the lesion.

[Patent literature 1] Japanese Published Unexamined Patent Application No. 2001-293016 (FIG. 1 on page 1)

Drug solutions including halogen elements such as fluorine, iodine or chloride were used as those permeated into a lesion in treatment by conventional iontophoresis-based medical devices. Drug solutions including metal elements such as silver or zinc were also used.

However, as described above, the conventional iontophoresis-based medical device was provided with a needle-shaped positive electrode section directly contacting with a lesion. Such a shape of the positive electrode section provided a lesion with a limited quantity of a drug solution at one time. Therefore, in order to attain a predetermined therapeutic effect, it was necessary to discontinue treatment to apply the drug solution to the positive electrode section several times, thus resulting in a prolonged treatment.

Further, according to the conventional iontophoresis-based medical device, the negative electrode section was designed to be attached to a part of the body largely apart from the lesion, for example, the wrist. Thus, the negative electrode section was a great distance from the positive electrode section and unable to give a sufficient iontophoresis-based therapeutic effect to the lesion.

In addition, the negative electrode section was required to have a dimension sufficient to be worn on the wrist. Further, in order to detachably attach the negative electrode section to the wrist conveniently, such an attachable and detachable structure as a spring-mounted supporting structure or belt-fastening structure was needed, entailing troublesome wearing and removing processes. Consequently, it was difficult to handle these electrode sections at the time of treatment.

In addition, conventional drug solutions were restricted to either drug solutions containing halogen elements or those containing metallic elements. Iontophoresis methods using these drug solutions were low in sterilization effect and none of them were able to attain a sufficient sterilization and treatment.

### SUMMARY OF THE INVENTION

Under these circumstances, the inventor discovered that current level necessary for treatment could be conducted even at a lower voltage by making a positive electrode and a negative electrode approximate to each other as much as possible to clip a lesion so that a current-conducting circuit was restricted to a narrow area and lowering an electric resistance in the circuit.

Finding that such arrangement was able to attain iontophoresis wherein drug ions were supplied only to a necessary area of a lesion, thus reducing to the least possible extent development of stimulation, derived adverse effects resulting in permeation of drug ions into cells and tissues other than necessary sites, the inventor completed the present invention. It was also found that use of the device at the lowest possible voltage was able to suppress electric stimulation to nerves.

An object of the invention is to provide an iontophoresis-based medical device which is able to increase a quantity of the drug solution supplied to a lesion, thereby reducing treatment time, improve the therapeutic effect of the lesion and also improve the handling of the positive electrode section and the negative electrode section during the treatment.

Further, this invention is to provide an iontophoresis-based medical device that is able to give treatment suitable for a position and condition of the lesion.

An additional object of the invention is to provide an iontophoresis-based medical device that is able to give a high sterilization effect to a lesion at a reduced cost through utilization of iontophoresis.

A further object of the invention is to provide an iontophoresis-based medical device that is able to provide a highly safe sterilization treatment.

The first aspect of the invention is an iontophoresis-based medical device comprising a positive electrode section capable of retaining a drug solution, a negative electrode section capable of retaining a solution, a power source supplying an electric current to the above-mentioned positive electrode section and negative electrode section and a controller controlling the current value and conduction time of electric current supplied from the power source, the iontrophoresis-based medical device for allowing the drug solution to permeate into a lesion based on ionphoresis obtained by conducting current between the above-mentioned positive electrode section and negative electrode section, wherein the above-mentioned positive electrode section and negative electrode section are respectively given a handleable stick shape, the above-mentioned positive electrode section is provided with a drug solution retainer which retains the above-mentioned drug solution and contacts with a lesion, and the above-mentioned negative electrode section is provided with a solution retainer which retains the above-mentioned solution and contacts with a part of the body in the vicinity of the lesion.

The iontophoresis-based medical device is to be used in treating humans or animals. A lesion is not restricted as long as it is a part of a human body that can contact with the positive electrode section.

The dimension and shape of the positive electrode section and the negative electrode section are not restricted as long as these can be handled. These are available, for example, as a stick with such a dimension and shape that can be held with one hand.

The drug solution is a solution in which a predetermined drug (medicine) is dissolved by water. The drug is not restricted to types, and, for example, halogen elements such as fluorine, iodine and chloride may be used. Metallic ions such as silver and zinc may also be used. In addition, a cationic surface active agent may be used.

Alternatively, other antimicrobial agents or antibiotics may be used as long as these are compounds (drugs) that can be dissolved in water and ionized as an ion.

No restrictions are given to methods for retaining a drug solution in the positive electrode section and those for retaining a solution in the negative electrode section. Further, such arrangement may be also acceptable where a large quantity of the drug solution or the solution is supplied to the positive electrode section and an excessive quantity of the drug solution or the solution is supplied to a lesion. In this instance, these solutions may be supplied continuously or intermittently.

The solution is not restricted to types, but preferable is a solution capable of increasing an electric conductivity. For example, sodium chloride solution (saline solution) potassium chloride solution, alum solution and calcium chloride solution may be used.

The drug solution retainer is not restricted to materials, shapes or dimensions, as long as the drug solution can be retained. The drug solution retainer is not restricted either to a site to be fixed on the positive electrode section, and can be fixed, for example, at the tip end of the positive electrode section.

The solution retainer is not restricted to materials, shapes or dimensions, as long as the solution can be retained. The solution retainer is not restricted to a site to be fixed on the negative electrode section either, and can be fixed, for example, at the tip end of the negative electrode section.

The power source may be a direct-current power source or an alternating current power source.

Upon electric conduction, the voltage value is to be 5V or lower, the current value is to be 40 μA or lower and the conduction time is to be 8 to 30 seconds. More particularly, in a case where dental pulp is sterilized and treated, for example, it is safe to apply a voltage value of less than 1.5V, a current value of less than 20 μA and an electric conduction time of less than 10 seconds. However, the current value and the conduction time should be determined, with consideration given to local conditions of the target site in the body.

An intensity of electric current is preferably 40 μA or lower, more preferably in a range from 20 to 40 μA. It is highly likely that an electric current exceeding 40 μA may cause drug-related damage (adverse effects) in the body. Further, an approximately constant current value corresponding to each target tissue makes it possible to control the action of drug solution ions only by adjusting the electric conduction time at subsequent processes. As a result, it is possible to treat a lesion in a simpler operation.

The electric conduction time is preferably 8 to 30 seconds and more preferably in a range from 15 to 30 seconds. A time of less than 8 seconds does not provide a sufficient therapeutic effect. Further, a time exceeding 30 seconds may result in development of damage to target tissues due to drug stimulation. However, in treating dental pulp, it is preferable to lessen the current value and the electric conduction time.

Further, the controller can be made up mainly with software by using a personal computer which is in the public domain, in addition to fabrication with electric circuits and elements mounted thereon.

The second aspect of the invention is the iontophoresis-based medical device of the first aspect of the invention wherein the above-mentioned drug solution retainer is provided with a mouth piece provided on the tip end of the stick in an attachable and detachable manner and a brush fixed on the mouth piece and the above-mentioned solution retainer is provided with a cylindrical head provided on the tip end of the stick and a sponge provided on the cylindrical head in an attachable and detachable manner.

The brush is not restricted to materials. For example, synthetic resin fur, animal fur and plant fur may be used as the materials. The sponge is not restricted to materials either. For example, urethane resin, polyethylene and polypropylene may be adopted as the materials. A cloth includes woven fabric, non-woven fabric and mesh cloth. Of these, brush is most preferable as the drug solution retainer. Cotton and sponge are most preferable as the solution retainer since these can be used excellently and supplemented with materials most easily available in clinical practices.

Further, the mouth piece is not restricted to configurations and can be formed as a shallow-bottom cylinder with an opening on one end, for example.

The third aspect of the invention is the iontophoresis-based medical device of the first aspect of the invention wherein the above-mentioned controller is able to set the current value, voltage value and electric conduction time on the above-mentioned electric conduction in response to the type of the target viscous membrane and thickness of the target skin and area of the target skin at the above-mentioned lesion.

The fourth aspect of the invention is the iontophoresis-based medical device of the first aspect having an alarm indicating the progress of the above-mentioned electric conduction time set by the above-mentioned controller.

An alarm (warning means) may include a buzzer and lighting.

The fifth aspect of the invention is the iontophoresis-based medical device of the first aspect of the invention wherein a main ingredient of the above-mentioned drug solution is a cationic surface active agent or an amphoteric surface active agent.

The cationic surface active agent includes, for example, benzalkonium chloride, benzethonium chloride, chlorhexidine gluconate and cation chloride cetylpyridinium, and the amphoteric surface active agent includes alkyldiaminoethylglycine hydrochloride.

Benzalkonium chloride is also known as alkyldimethylbenzylammonium salt and a sterilizing agent, which is aliphatic quaternary ammonium salt and available at a reasonable cost. This agent is listed in the Japanese Pharmacopoeia. It is a colorless or pale yellow aqueous solution and obtained by allowing alkyldimethylamine to react with benzylchloride.

Benzethonium chloride is also known as benzyldimethyl {2-[2-(p-1,1,3,3-tetramethylbutylphenoxy)etoxy]ethyl}ammonium chloride and a sterilizing agent listed in the Japanese Pharmacopoeia. This agent exhibits an antimicrobial activity against a wide range of bacteria and fungi, having a cleaning effect, keratolytic effect and emulsion effect.

The sixth aspect of the invention is the iontophoresis-based medical device of the second aspect of the invention wherein a 1 to 3% sodium chloride solution is impregnated into the above-mentioned sponge.

The seventh aspect of the invention is the iontophoresis-based medical device of the first aspect of the invention wherein the above-mentioned lesion is an oral lesion such as periodontal tissue, teeth, dental pulp or root canal or a superficial lesion on the body.

Dental disorders found as a lesion include periodontal disease, pulpitis and infected root canal.

Superficial lesions include skins which have developed candidiasis and ringworm.

The eighth aspect of the invention is the iontophoresis-based medical device of the seventh aspect of the invention wherein the above-mentioned current value is 40 μA or lower and the electric conduction time is 8 to 30 seconds when the above-mentioned lesion is an oral lesion in humans.

The ninth aspect of the invention is a method for sterilizing and disinfecting body tissues by using iontophoresis wherein a drug solution retained by a positive electrode section is allowed to come into contact with an oral lesion in body tissues and a solution retained by a negative electrode section is allowed to come into contact with the vicinity of the above-mentioned lesion, thereby providing an electric closed circuit between these electrode sections and the lesion to conduct a current of 40 μA or lower into the closed circuit for 8 to 30 seconds.

The tenth aspect of the invention is a method for sterilizing and disinfecting body tissues by using iontophoresis of the ninth aspect of the invention wherein a main ingredient in the above-mentioned drug solution is a cationic surface active agent or an amphoteric surface active agent.

The eleventh aspect of the invention is a method for sterilizing and disinfecting body tissues by using iontophoresis of the ninth aspect of the invention wherein the above-mentioned solution is sodium chloride solution.

The twelfth aspect of the invention is a method for sterilizing and disinfecting body tissues by using iontophoresis wherein a drug solution retained by a positive electrode section is allowed to come into contact with a superficial lesion in a human body or small animals and a solution retained in a negative electrode section is allowed to come into contact with the vicinity of the above-mentioned lesion, thereby providing a closed electric circuit between these electrode sections and the lesion to conduct a current of 0.2 to 0.5 mA into the closed circuit for a predetermined time.

The thirteenth aspect of the invention is a method for sterilizing and disinfecting body tissues by using iontophoresis of the twelfth aspect of the invention wherein current value, voltage value and electric conduction time on the above-mentioned electric conduction are set in response to the thickness and area of the target skin at the above-mentioned lesion.

According to this invention, for example, an operator allows a drug solution retainer in which a drug solution is retained to come into contact with a lesion (part of the body tissue), with a positive electrode section held with one hand, and also allows a solution retainer in which a solution is retained to come into contact with the lesion or a part of the body around the lesion (for example, a site as close as possible to the lesion), with a negative electrode section held with the other hand, thereby providing a closed electric circuit among a power source, the electrode sections and the lesion (including the site around the lesion). When a low electric current is conducted from the power source, in this condition, iontophoresis takes place, allowing the drug solution to permeate deeply into the lesion. At this time, a large quantity of the drug solution retained by the drug solution retainer is present in the positive electrode section and a large quantity of the solution retained by the solution retainer is present in the negative electrode section, thereby further increasing the iontophoresis effect than that obtained by a conventional method. Therefore, a lesion can be treated including that at a deep site, the treatment of which is otherwise difficult by mere application of a drug solution.

As explained above, since a large quantity of the drug solution can be retained in the drug solution retainer, it is possible to supply the drug solution to a lesion in a greater quantity than by a device provided with a positive electrode section having a conventional needle shape. Thus, the treatment time can be shortened and a lesion can be treated more effectively. Further, since the negative and positive electrode sections are given a stick shape comprised of a bar or a shaft having a predetermined length and thickness that can be handled, the electrode sections can be improved in handling at the time of treatment.

In addition to an antimicrobial effect derived from the iontophoresis action, an electric field is imparted to an aqueous solution to develop a bactericidal action directly from the electric field. Minute electrolytes contained in water combine or collide with a negative electric charge based on dissociation of phosphate groups existing on nucleotides of organisms or viruses, thus breaking nucleic acids to attain an antimicrobial effect. The minute electrolytes contained in water include positively charged ions such as H₃O⁺ or H₉O⁺ resulting from hydronium ions produced by electrolysis of sodium chloride or others.

Controlling the voltage supplied to an electric circuit, current value and electric conduction time in particular by the controller makes it possible to provide treatment suitable to positions and conditions of the lesion.

Cationic surface active agent and amphoteric surface active agent are higher in an antimicrobial effect than a conventional drug such as halogen elements and metallic elements. Consequently, the lesion to which these surface active agents are permeated can be favorably sterilized.

Since drug solutions in which a cationic surface active agent or amphoteric surface active agent is used as a main ingredient are used as drug solutions for iontophoresis, it is possible to give a potent antimicrobial effect to a lesion by utilizing iontophoresis at a lower cost.

### BRIEF DESCRIPTION OF THE DRAWINGS

FIG. 1 is an overall perspective view of the iontophoresis-based medical device according to one embodiment of the present invention.

FIG. 2(a) is a sectional view of the positive electrode section used in the iontophoresis-based medical device according to one embodiment of the present invention. FIG. 2(b) is an exploded sectional view of the positive electrode section used in the iontophoresis-based medical device according to one embodiment of the present invention.

FIG. 3(a) is a sectional view of the negative electrode section used in the iontophoresis-based medical device according to one embodiment of the present invention. FIG. 3(b) is an exploded sectional view of the negative electrode section used in the iontophoresis-based medical device according to one embodiment of the present invention.

FIG. 4 is an electric circuit diagram of the controller of the iontophoresis-based medical device according to one embodiment of the present invention.

FIG. 5 is a plan view of a status of sterilization and treatment in the vicinity of a molar by the iontophoresis-based medical device according to one embodiment of the present invention.

FIG. 6(a) is a side elevation view explaining how to use the positive electrode section in the vicinity of a molar. FIG. 6(b) is a side elevation view explaining how to use the negative electrode section in the vicinity of the molar.

FIG. 7 is a sectional view showing a status of the sterilization and treatment of the molar by the iontophoresis-based medical device according to one embodiment of the present invention.

FIG. 8 is a sectional view showing a status of the sterilization and treatment of a front tooth by the iontophoresis-based medical device according to one embodiment of the present invention.

### DETAILED DESCRIPTION OF THE PREFERRED EMBODIMENT

The embodiment of the present invention will be explained hereinafter by referring to the drawings.

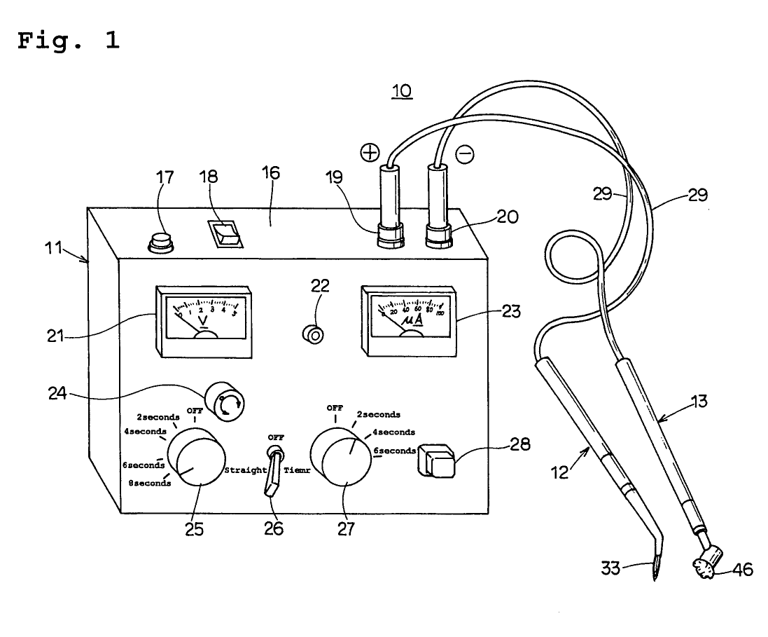

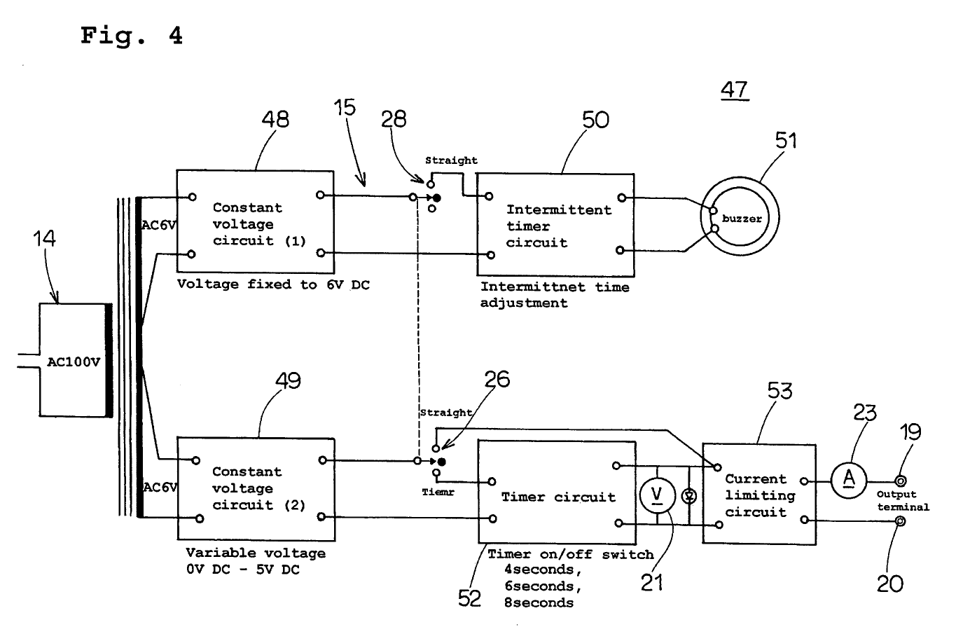

In FIG. 1 and FIG. 4, 10 denotes an iontophoresis-based medical device according to one embodiment of the present invention, the iontophoresis-based medical device 10 comprises a device body 11, a stick-shaped positive electrode section 12 connected to the device body 11 and capable of retaining a drug solution in which an anti-microbial drug solution is dissolved, a stick-shaped negative electrode section 13 connected to the device body 11 and capable of retaining a solution increasing the conductivity, and an electric circuit 15 connecting the positive electrode section 12 and the negative electrode section 13 respectively with a power source 14.

The device body 11 is provided with a rectangular device box 16. On the top of the device box 16, mounted are a fuse 17, a power switch 18, a positive electrode connecting terminal 19 and a negative electrode connecting terminal 20. Further, on the front plate of the device box 16, mounted are a voltage indicator 21, a pilot lamp 22, an ampere meter 23, a voltage adjusting knob 24, a buzzer knob 25 which changes the buzzer (alarm) sounding time and is built into the device box 16, an output switch 26 for changing from continuous conduction to timer control or vice versa, an electric conduction time knob 27 for changing the electric conduction time, and a timer start switch 28.

The positive electrode connecting terminal 19 is connected through a lead wire 29 to the positive electrode section 12. Further, the negative electrode connecting terminal 20 is connected through a lead wire 29 to the negative electrode section 13. The power source 14 is a 100V alternate current for home use, which is converted into direct current (6V) through a converter upon usage.

The buzzer knob 25 is a knob for adjusting intervals of sounding of the buzzer indicating a passage of predetermined electric conduction time (sterilization and treatment time). Intervals of the buzzer can be arbitrarily selected from four different intervals of 2 seconds, 4 seconds, 6 seconds and 8 seconds. It is also possible to make such an arrangement that the alarm lamp can be made to flash according to the sound of the buzzer.

Next, electric conduction time knob 27 is a knob for adjusting intervals of switching between conduction and discontinued conduction. The conduction and discontinued conduction can be arbitrarily selected from three intervals of 2 seconds, 3 seconds and 6 seconds.

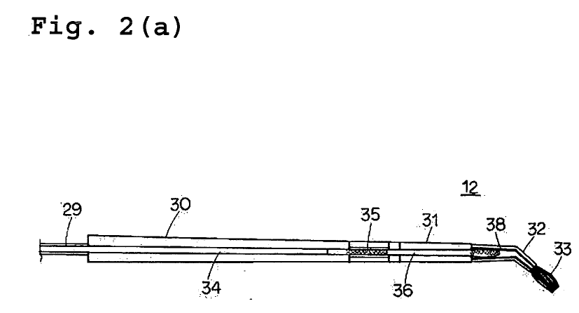

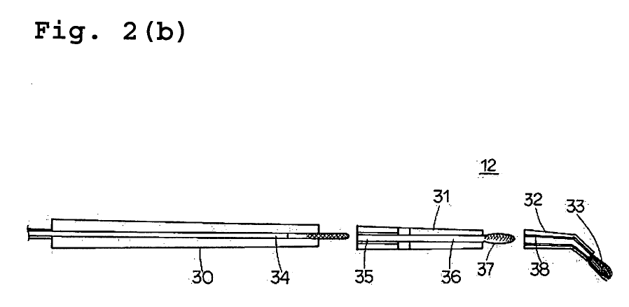

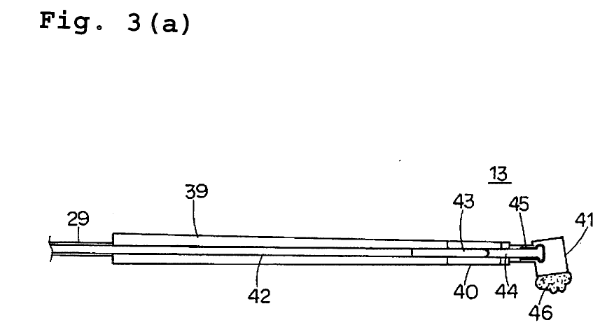

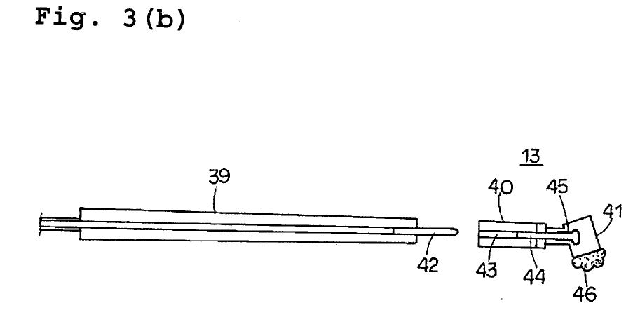

Then, an explanation will be made for the positive electrode section 12 and the negative electrode section 13, by referring to FIG. 2 and FIG. 3.

The positive electrode section 12 shown in FIG. 2 is mainly provided with a handle 30 which is an insulative plastic-made narrow tube, an intermediate part 31 which is an insulative plastic-made narrow tube connected to the tip end of the handle 30 in an attachable and detachable manner and having about half the length of the handle 30, a brass-made mouth piece 32 connected to the tip end of the intermediate part 31 in an attachable and detachable manner, the end of which is bent about 45 degrees, and an animal fur-fabricated writing brush (drug solution retainer) 33 fixed at the tip end of the mouth piece 32.

A nickel silver wire 34 connected to a lead wire 29 extended from said positive electrode connecting terminal 19 is inserted into a duct of the handle 30 which is a pipe having a circular cross section. The tip end of the nickel silver wire 34 protrudes from the end surface of the handle 30. A narrow nickel silver tube 35 is fitted into a duct on the proximal side of the intermediate part 31. A nickel silver wire 36 connected with a nickel silver tube 35 is inserted into the other duct of the intermediate part 31. When the handle 30 is connected with the intermediate part 31, the tip end of the nickel silver wire 34 on the side of the handle 30 is attached to the proximal side of the nickel silver tube 35. A gold-plated 37 tip end of the nickel silver wire 36 protrudes from the end surface of the intermediate part 31. An inner wall of the duct of the mouth piece 32 is provided with gold plate 38 contacting with said writing brush 33 on the tip end. Benzalkonium chloride solution is impregnated into the writing brush 33. When the intermediate part 31 is connected with the mouth piece 32, the tip end of a nickel silver wire 36 on the side of the intermediate part 31 is inserted into the proximal part of the mouth piece 32. Thus, connecting the handle 30, the intermediate part 31 and the mouth piece 32 can electrically connect the positive electrode connecting terminal 19 of the device body 11 with the writing brush 33.

The negative electrode section 13 shown in FIG. 3 is mainly provided with a handle 39 which is an insulative plastic-made narrow tube, an intermediate part 40 which is an insulative plastic-made short and narrow tube connected to the tip end of the handle 39 in an attachable and detachable manner and a short cylindrical head 41 fixed to the tip end of the intermediate part 40.

A nickel silver wire 42 connected to a lead wire 29 extending from said negative electrode connecting terminal 20 is inserted into a duct of the handle 39. The tip end of the nickel silver wire 42 protrudes from the end surface of the handle 39. A narrow nickel silver tube 43 is fitted into a duct on the proximal side of the intermediate part 40, and a nickel silver wire 44 connected with the nickel silver tube 43 is inserted into the other duct of the intermediate part 40. When the intermediate part 40 is connected with the handle 39, the tip end of the nickel silver wire 42 on the side of the handle 39 is attached to the proximal side of the nickel silver tube 43. The nickel silver wire 44 on the side of the intermediate part 40 is gold-plated 45 at the tip end, and extended up to the inside of the cylindrical head 41. The cylindrical head 41 is made with polypropylene and provided with a sponge (solution retainer) 46 on the top of the cylindrical head in an attachable and detachable manner. For increasing the conductivity, 1 to 3% (% by volume) sodium chloride aqueous solution is impregnated into the sponge 46. Thus, connecting the handle 39 with the intermediate part 40 to which the cylindrical head 41 is attached can provide an electric connection of the negative electrode connecting terminal 20 with the sponge 46 in the device body 11.

Now, an explanation will be made for the controller 47 of the iontophoresis-based medical device 10 by referring to FIG. 4.

As shown in FIG. 4, the controller 47 acts to convert a 100V alternate current into a 6V direct current through a rectifier-built constant voltage circuit 48 and also converts it to 0 to 5V direct current through a trans- and rectifier-built constant voltage circuit 49. The constant voltage circuit 48 supplies direct current for an intermittent timer circuit 50 to actuate a buzzer 51. The buzzer 51 is directly connected to the intermittent timer circuit 50. The constant voltage circuit 49 supplies direct current to a timer circuit 52 for conduction. A current limiting circuit 53 is connected to a timer circuit 52. Said positive electrode connecting terminal 19 and the negative electrode connecting terminal 20 are respectively connected to the current limiting circuit 53.

Next, an explanation will be made of the method for sterilizing and treating teeth or gingivae by using the iontophoresis-based medical device 10, with reference to FIG. 1 and FIG. 5 through FIG. 8.

First, 3% benzalkonium chloride aqueous solution is impregnated into the writing brush 33 and 3% sodium chloride aqueous solution is also impregnated into the sponge 46. Thereafter, the power switch 18 is turned on and the voltage adjusting knob 24 is turned to set the voltage to 1.5 to 2.0V. Further, the output switch 26 is turned to make an arbitrary selection, namely, sterilization and treatment by using continuous conduction or that by controlling the electric conduction time based on the buzzer 51 or controlling the conduction time based on the automatic on/off switch. In this instance, when the control based on the buzzer or electric conduction time is selected, the corresponding buzzer knob 25 or the electric conduction time knob 27 is turned to set a time until the buzzer 51 is sounded or intervals of switching from electric conduction to a lesion to discontinuation of the conduction.

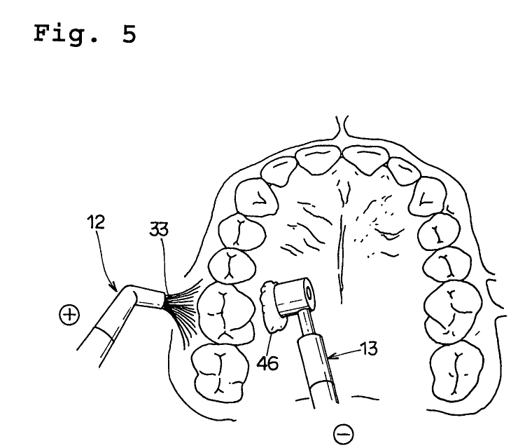

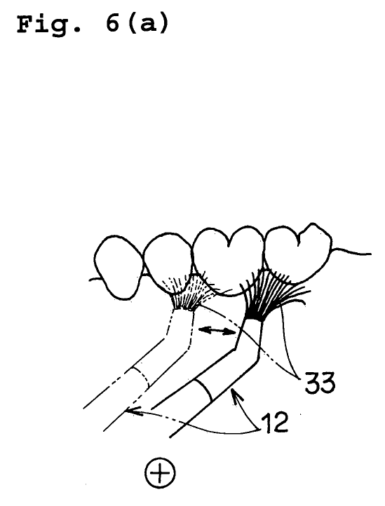

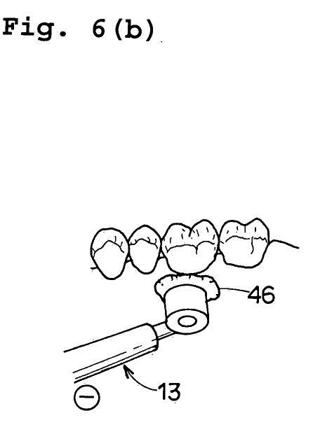

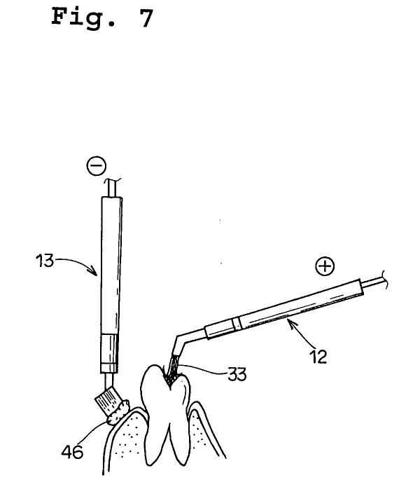

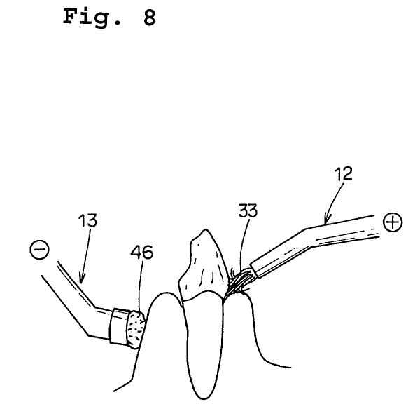

Then, the writing brush 33 retaining benzalkonium chloride solution in the positive electrode section 12 is allowed to contact with a tooth or a part of the gingiva infected by a pathogen (FIG. 5, FIG. 6(a), FIG. 7 and FIG. 8). In addition, the sponge 46 to which sodium chloride aqueous solution of the negative electrode section 13 is impregnated is allowed to contact with the oral tissue in the vicinity of the above-mentioned infected tooth or gingiva (FIG. 5, FIG. 6(b), FIG. 7 and FIG. 8), thereby providing a closed electric circuit among the constant voltage circuit 49, current limiting circuit 53, positive and negative electrode sections 12 and 13, affected tooth, affected gingiva and oral tissue around the lesion. While maintaining this condition, the timer start switch 28 is turned on and a low current of 40 μA is conducted from the constant voltage circuit 49 through the current limiting circuit 53. Then, benzalkonium chloride is impregnated deeply into the tooth as ion through the action of iontophoresis. In this instance, a large quantity of benzalkonium chloride aqueous solution retained in the writing brush 33 is present in the positive electrode section 12 and a large quantity of sodium chloride aqueous solution retained in the sponge 46 is present in the negative electrode section 13, thereby offering a higher effect of iontophoresis than a device having the conventional needle-shaped electrode sections. Further, benzalkonium chloride is higher in an antimicrobial effect than conventional antimicrobial drugs such as halogen elements and metallic elements. Consequently, benzalkonium chloride can permeate deeply into a lesion and result in an effective sterilization of the tooth. The current value and electric conduction time should be appropriately determined, for example, in the respective ranges of 20 to 40 μA and 8 to 30 seconds, with consideration given to the position and condition of the target lesion.

In addition to an antimicrobial effect derived from the iontophoresis action, an electric field is imparted to an aqueous solution to develop a bactericidal action directly from the electric field. Minute electrolytes contained in water, namely, positively charged ions such as H₃O⁺ or H₉O⁺ resulting from hydronium ions produced by electrolysis of sodium chloride and others, combine or collide with negative electric charge based on dissociation of phosphate groups existing on nucleotides of organisms or viruses to break nucleic acids, attaining an antimicrobial effect.

As explained above, since a large quantity of the benzalkonium chloride solution can be retained in the writing brush 33, it is possible to supply the drug solution to a lesion in a greater quantity than by a device provided with a positive electrode section having a conventional needle shape. Thus, the treatment time can be shortened and a lesion can be treated more effectively. Further, since the positive and negative electrode sections 12 and 13 are given a handleable shape, these electrode sections, 12 and 13, can be improved for handling at the time of treatment.

Further, since solutions used in iontophoresis include those in which benzalkonium chloride is contained as a major active ingredient, it is possible to give a potent antimicrobial effect to an affected tooth or an affected gingiva by utilizing iontophoresis at a lower cost.

In addition, controlling the current value and electric conduction time by the controller 47 makes it possible to provide treatment suitable to positions and conditions of the lesion.

Now, an explanation will be made about the relationship between the current value and the electric conduction time on the above conduction for a target to be treated.

It is recommended to employ a current density smaller than 1 mA per square inch (=6.25 square cm) for treatment purposes. A current density of lower than 40 μA is preferable for a target area of 5 mm×5 mm.

In particular, for the purpose of dental treatment, the current density depends on an intensity of a positive electrode for each tooth, preferably not exceeding 40 μA in the positive electrode with the size of a small writing brush. More particularly, the current density is fixed to be 40 μA at the power source voltage of 1.5 to 2.5V, and controls the electric conduction time. For example, electric conduction is carried out for 20 to 30 seconds on the buccal side and similarly on the lingual side.

Further, for the purpose of dermatologic treatment, it is necessary to use an appropriate positive electrode according to the target skin area (a larger positive electrode is used for a larger area of the target lesion). In other words, when 7V of a power source voltage is used, an appropriate current value ranges from 0.2 to 0.5 mA and the electric conduction time is adjusted accordingly. Treatment based on similar voltage and current is carried out in treating diseases of small animals.

In addition, it is necessary to make an appropriate selection of the voltage, current and electric conduction time depending on the thickness and area of mucous membrane and skin in providing sterilization and treatment.

According to the invention, such an arrangement that a drug solution retainer capable of retaining a large quantity of a drug solution is provided in the positive electrode section makes it possible to supply the drug solution in a greater quantity to a lesion than a device having a conventional needle-shaped positive electrode section. Thus, the treatment time can be shortened and a lesion can be treated more effectively. Further, since the positive and negative electrode sections are given a handleable stick or shaft shape, these can be improved for handling of both electrode sections at the time of treatment.

Controlling the current supplied to an electric circuit and electric conduction time in particular makes it possible to provide sterilization and treatment appropriate to the position and conditions of a lesion.

In addition, since drug solutions in which a cationic surface active agent or amphoteric surface active agent is used as a main ingredient are used as drug solutions for treatment utilizing iontophoresis, it is possible to give a potent antimicrobial effect to a lesion at a lower cost. Use of benzalkonium chloride or benzethonium chloride also provides other economic advantages.

What is claimed is:

1. An iontophoresis-based medical device for an animal comprising:
a positive electrode section capable of retaining a drug solution;
a negative electrode section capable of retaining a solution;
a power source supplying an electric current to the positive electrode section and negative electrode section; and
a controller controlling a value and an electric conduction time of the electric current supplied from the power source, thus allowing the drug solution to permeate into a lesion based on ionphoresis obtained by conducting the electric current between the positive electrode section and negative electrode section, wherein
the positive electrode section and the negative electrode section are respectively given a handleable stick shape,
the positive electrode section is provided with a drug solution retainer which is configured to retain a large quantity of the drug solution and contacts with the lesion, and
the negative electrode section is provided with a solution retainer which is configured to retain a large quantity of the solution and contacts with a part of the body in the vicinity of the lesion,
the drug solution retainer of the positive electrode section and the solution retainer of the negative electrode section are made of different materials from each other such that the drug solution retainer is made of a writing brush and the drug solution is made of a sponge, and
the drug solution is a solution containing an amphoteric surface active agent having a sterilizing and disinfecting effect as a main ingredient, and the second solution is a sodium chloride solution having a concentration of 1 to 3%.

2. The iontophoresis-based medical device for an animal according to claim 1, wherein the drug solution retainer is provided with a mouthpiece which is provided at the tip end of the stick in an attachable and detachable manner and to which the writing brush is fixed on the mouth piece, and the solution retainer is provided with a cylindrical head which is provided at the tip end of the stick and to which the sponge is provided at the cylindrical head in an attachable and detachable manner.

3. The iontophoresis-based medical device for an animal according to claim 1, wherein the controller is able to set a current value, a voltage value and an electric conduction time for the conduction in response to type of a target viscous membrane, a thickness of a target skin and an area of the target skin at the lesion.

4. The iontophoresis-based medical device for an animal according to claim 1, further comprising an alarm indicating the progress of the electric conduction time set by the controller.

5. The iontophoresis-based medical device for an animal according to claim 1, wherein the lesion is an oral lesion such as a periodontal tissue, teeth, dental pulp or a root canal or a superficial lesion on the body.

6. The iontophoresis-based medical device for an animal according to claim 5, wherein the current value is 40 μA or lower and the electric conduction time is 8 to 30 seconds when the lesion is an oral lesion in humans.

7. A method for sterilizing and disinfecting a body tissue of an animal using iontophoresis comprising the steps of:
contacting an oral lesion in the body tissue with a drug solution of an animal retained and supplied by a drug solution retainer of a positive electrode section;
contacting a body tissue in the vicinity of the lesion with a solution retained and supplied by a solution retainer of a negative electrode section, to provide a closed electric circuit between the electrode sections and the lesion; and
conducting an electric current into the closed circuit, wherein
the drug solution retainer of the positive electrode section and the solution retainer of the negative electrode section are made of different materials from each other such that the drug solution retainer is made of a writing brush and the drug solution is made of a sponge, and
the drug solution is a solution containing an amphoteric surface active agent having a sterilizing and disinfecting effect as a main ingredient, and the second solution is a sodium chloride solution having a concentration of 1 to 3%.

8. A method for sterilizing and disinfecting a body tissue of an animal using iontophoresis comprising the steps of:
contacting a superficial lesion of an animal with a drug solution retained and supplied by a drug solution retainer of a positive electrode section;
contacting a body tissue in the vicinity of the lesion with a solution retained and supplied by a solution retainer of a negative electrode section, to provide a closed electric circuit between the electrode sections and the lesion; and
conducting an electric current into the closed circuit for a predetermined time, wherein
the drug solution retainer of the positive electrode section and the solution retainer of the negative electrode section are made of different materials from each other such that the drug solution retainer is made of a writing brush and the drug solution retainer is made of a sponge, and
the drug solution is a solution containing an amphoteric surface active agent having a sterilizing and disinfecting effect as a main ingredient, and the second solution is a sodium chloride solution having a concentration of 1 to 3%.
